(19) United States
(12) Patent Application Publication
(10) Pub. No.: US 2017/0273823 A1
(43) Pub. Date: Sep. 28, 2017

(54) SYSTEM FOR PROVIDING INTERVAL THERMAL THERAPY

(71) Applicant: MG Therapies, Inc., Buffalo, WY (US)

(72) Inventors: Donald James Novkov, Encinitas, CA (US); David Michael McMahon, Del Mar, CA (US)

(73) Assignee: MG Therapies, Inc., Buffalo, WY (US)

(21) Appl. No.: 15/467,992

(22) Filed: Mar. 23, 2017

Related U.S. Application Data
(60) Provisional application No. 62/312,413, filed on Mar. 23, 2016.

(51) Int. Cl. A61F 7/00; A61F 7/02

(52) U.S. Cl. CPC A61F 7/007; A61F 7/02; A61F 2007/0075; A61F 2007/0004; A61F 2007/0087; A61F 2007/0296

(57) ABSTRACT

The disclosure concerns a computer-controlled system for interval thermal therapy; the system including a control unit console configured for placement on a bench-top or similar setup, the console is connected to a hand-held thermal therapy application assembly, either by way of a cable extending between the hand-held assembly and the console, or via a cordless coupling therebetween. The hand-held assembly generally comprises a removeably applicator with a thermally conductive contact surface configured to communicate hot or cold thermal energy, or a combination of hot and cold thermal therapy in various intervals, the contact surface of the hand held assembly is used to contact a patient's body at a desired treatment site for effectuating various physiological treatments or therapies.

### CROSS-REFERENCE TO RELATED APPLICATIONS

This application claims benefit of U.S. Provisional Ser. No. 62/312,413, filed Mar. 23, 2016; the entire contents of which are hereby incorporated by reference.

### BACKGROUND

Field of the Invention

The invention in its various embodiments relates to bench-top, electrical medical equipment; and more particularly, to such equipment configured to provide interval thermal therapy for medical applications.

Description of the Related Art

There is a need for a single system capable of independently, selectively and/or programmatically administering hot or cold thermal therapy, or modulations of combination hot and cold thermal therapy, to various tissue regions associated with the body of a human or animal patient using a single system capable of providing such thermal therapy without changing instruments in between a thermal therapy session.

In addition, there is a need for a system configured to contact a variety of tissue regions (such as eyelids, cheeks, forehead, arms, etc.) with an adjustable or selectable contact surface; i.e. a surface having an adjustable or selectable surface contour or shape, such that the contact surface can be varied depending on the treatment site specifications, or the variation of size and shape of the treatment site associated with a particular individual patient with respect to others.

Further, there is a need to provide consistent and repeatable treatment, including a consistent thermal therapy, or a modulated thermal therapy, for a precise duration or interval program, such that results can be repeatedly achieved and progression of the treatment effectively monitored and investigated.

Finally, conventional systems for communicating thermal therapy generally utilize a fluid communicator. Problems associated with fluid-type thermal therapy devices include, but are not limited to: (i) fluid maintenance and replacement requirements, including the cost and labor associated therewith; (ii) cumbersome techniques for instrument sterilization; (iii) additional weight of the fluid and its impact on, inter alia, ergonomics; and (iv) propensity for leakage or spillage of the fluid.

These and other problems in the art are addressed by the disclosed embodiments of a system for providing thermal therapy as disclosed and claimed herein.

### SUMMARY

The invention concerns a computer-controlled system for interval thermal therapy; the system including a control unit console configured for placement on a bench-top or similar setup, the console is connected to a hand-held thermal therapy application assembly, either by way of a cable extending between the hand-held assembly and the console, or via a cordless coupling therebetween. The hand-held assembly generally comprises a removeable applicator with a thermally conductive contact surface configured to communicate hot or cold thermal energy, or a combination of hot and cold thermal therapy in various intervals, the contact surface of the hand held assembly is used to contact a patient's body at a desired treatment site for effectuating various physiological treatments or therapies.

In one embodiment, the system is used for the treatment of Meibomian Gland Dysfunction (MGD), or posterior blepharitis; a common physiological ailment related to the lack of proper flow of meibomian gland secretions, resulting in a condition known as “dry eye”.

The contact surface generally forms part of a removeable applicator, wherein one of a plurality of possible removeable applicators may be selected for insertion and integration with the hand-held assembly, such that a desired thermal therapy treatment may be administered.

Energy is imparted into the system for hot and/or cold therapy by way of a thermal electric cooler.

Other features and advantages of the system for providing interval thermal therapy are further disclosed in the appended detailed description of the preferred embodiments.

### BRIEF DESCRIPTION OF THE DRAWINGS

Now, the invention in its several embodiments may be further appreciated with reference to the drawings, wherein:

FIG. 1 shows a system for providing interval thermal therapy in accordance with a preferred embodiment;

FIG. 2A shows the system in accordance with the preferred embodiment being represented from an alternative viewing perspective;

FIG. 2B shows a rear perspective view of the system in accordance with the preferred embodiment;

FIG. 3 shows a hand-held assembly for communicating thermal therapy to a patient in accordance with one embodiment;

FIGS. 4(A-B) show a side view, and rear view, respectively, of the hand-held assembly for communicating thermal therapy in accordance with the embodiment of FIG. 3;

FIG. 5 shows a cross-section view of an engagement interface for engaging a releasable thermal energy applicator with a thermal conduction and engaging mechanism of the hand-held assembly;

FIG. 6 illustrates a cross section of a hand-held assembly in accordance with another embodiment, and further illustrates an arrangement of key components for achieving hot and cold thermal energy transfer;

FIG. 7 illustrates a cross section of a hand-held assembly in accordance with yet another embodiment, wherein hot therapy only is produced and communicated to an applicator interface.

FIG. 8 shows a removeable thermal energy applicator for use with the system, the applicator comprises a contact surface for communicating thermal energy to a treatment site of a patient;

FIGS. 9(A-B) show another removeable applicator for use with the system, the applicator comprises a debridement tool.

FIGS. 10(A-B) show another removeable applicator for use with the system, the applicator comprises a chalazion clamp.

FIGS. 11(A-B) show another removeable applicator for use with the system, the applicator comprises a paddles;

FIGS. 12(A-B) show the system with an integrated thermal energy applicator assembly, the applicator assembly comprises paddles for paddle expression, wherein the paddles are configured for actuation upon translation of a handle;

FIGS. 13(A-B) show the system with an integrated thermal energy applicator assembly, the applicator assembly comprises rollers for roller expression, wherein the rollers are configured for actuation upon translation of a handle;

FIG. 14 shows an example of a graphical user interface (GUI) for displaying on the display screen of the system, the GUI permits user-interaction for selecting and customizing a thermal therapy procedure;

FIG. 15 shows another example of the GUI associated with the system, wherein information specific to a patient and treatment procedure are input for storage; and

FIG. 16 shows a plot of an example therapy session which can be provided using the interval thermal therapy system.

### DETAILED DESCRIPTION OF EMBODIMENTS

The disclosure concerns a system configured to provide thermal therapy to a treatment site of a patient, the thermal therapy comprising communication of heat, or cold thermal therapy, or a combination thereof, and can be constant or modulated to provide interval thermal therapy procedures. Herein, the various embodiments provide a heated or cooled contact surface for purposes of medical treatment or physical therapy.

The main bench-top component (herein referred to as the “console”) responds to user commands via a computer Graphical User Interface (GUI) and provides power to and receives data from the hand-held thermal therapy application assembly (herein referred to as the “wand” or “hand-held assembly”). The console contains the computer, power supply and electronics, wherein the wand contains the thermal energy generation and communication components.

In certain embodiments, the wand comprises a Thermoelectric Cooler (TEC), sometimes referred to as a Thermoelectric Module (TEM), otherwise known as a Peltier, or more descriptively a Peltier device assembly, which includes: a plurality of alternating n-type and p-type semiconductors connected electrically in series, arranged such that their thermal output due to the Peltier effect is in parallel upon application of electrical current. The TEC is a two-wire device that is polarity sensitive as described further herein. The console provides electrical power to the wand to produce electrical current which is used to produce the desired heated or cooled surface.

The wand also contains one or more temperature sensors, such as a thermistor, thermocouple or resistance temperature detector (RTD) for the purpose of measuring the heated surface temperature for feedback into a temperature control system and for monitoring on the GUI. The wand may also contain a thermal cutoff fuse that is connected to the console electronics in such a manner as to cause power to the TEC to be cut, using a relay or other means, in the event of an electronics failure that causes excessive heating of the surface.

### System for Interval Thermal Therapy:

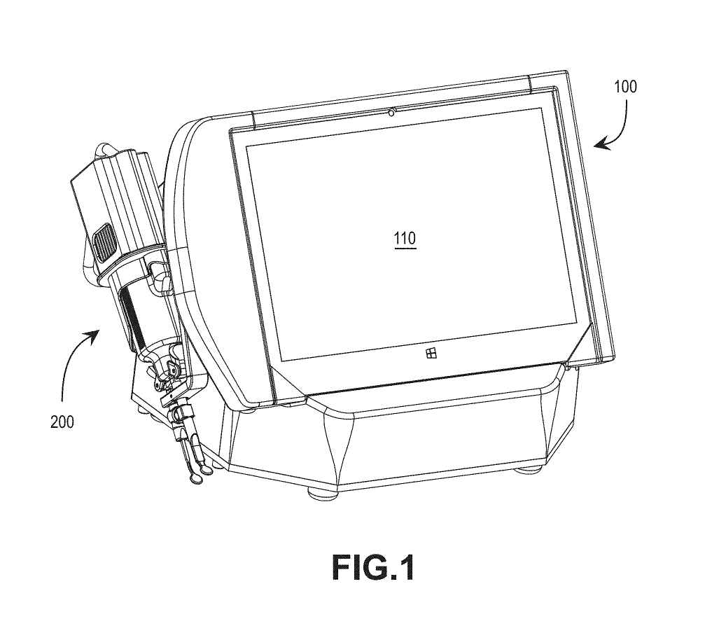

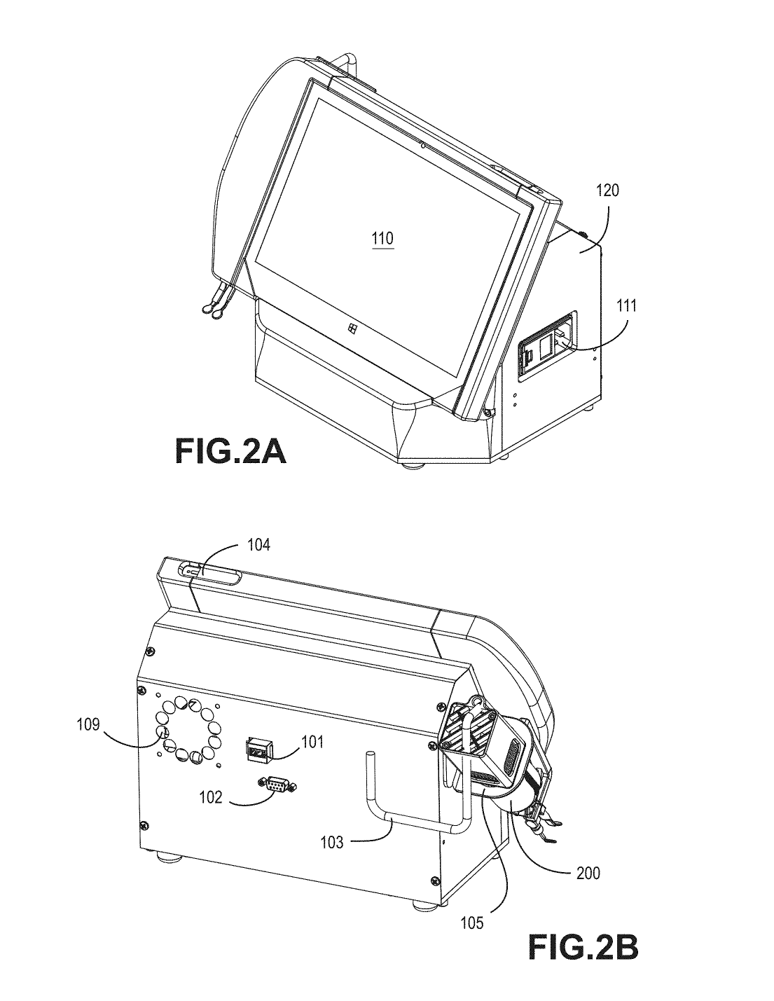

Referring now to FIGS. 1, and 2A-2B, the system for providing interval thermal therapy comprises: a computer console 100, the computer console including a display screen 110, a conventional computer assembly housed within a console housing 120, and a power entry assembly 111 for introducing power to the console; and a wand 200, including a removeable applicator and integrated thermal contact surface.

The console 100 may further comprise: a handheld-assembly bracket or mount 105 for storing the wand (as shown); a serial port 102, such as a DB-9 or similar connector or port; an external connector 101, such as a LAN or USB port or connector; a ventilation opening 109 for venting heat from the console as produced by the computer; a power activation switch 104; or a plurality or combination thereof. Other conventional features associated with computerized systems may be implemented in accordance with the ordinary level of knowledge and skill in the art.

The thermal energy applicator is removable from the wand. This allows it to be cleaned independently, for example autoclaved or using other sterilization techniques. More significantly, this allows for numerous physical configurations, shapes and sizes of interchangeable thermal energy applicators to be used in the same wand. Thus, for one particular use, such as Meibomian Gland Disorder treatment, the thermal energy applicator can be of a particular size and shape suitable for contacting the eyelid. As another example, the part size and shape can be suitable for treatment of another eye disorder or for use on a particular surface area of the body where a muscle targeted for treatment is located.

As discussed infra, a number of example thermal energy applicators are disclosed. However, those having skill in the art will be positioned to appreciate other non-illustrated thermal therapy applicators that can be used with the presently disclosed system for providing interval thermal therapy.

The computer assembly associated with the system and housed in the console may be a touch-screen or standard type, containing a standard microprocessor, operating system, motherboard, electronics and peripheral connectors commonly associated with touch-screen or tablet-type computers. All of the user commands may be sent using the touch-screen and all of the data presented for monitoring is presented on the touch-screen; or otherwise using a mouse or similar configuration. Alternatively, the commands may be provided via voice recognition or similar technique. An external connection to the computer may be provided allowing for a conventional keyboard and/or mouse to also be connected to allow for user commands to be sent via these standard interfaces.

Referring to FIG. 2B, the external computer connection 101 is represented as a single Universal Serial Bus (USB) connector, but could be any other type of computer communication connector such as Ethernet or parallel port and there may be multiple ports. Also, if this function is not built into the computer, a Wi-Fi or similar type of radio communication device may be connected to the computer housed within the console to supplant the external connection or otherwise to allow for an external computer to interface with the console computer. GUI software and microprocessor firmware may be uploaded to the device, and data may be logged and downloaded from the device using one of these connections, either wired or wirelessly.

Referring to FIGS. 2A and 2B, in one embodiment is shown the back cover of the console 100, behind which are housed the main power supply, a power entry module 111, electronics that interface with the wand 200, and a ventilation opening 109 that may or may not include a fan to produce forced ventilation (in conjunction with various other openings in the enclosure). In the embodiment shown, the power on/off switch for the input AC power is located on the power entry module 111. Not shown is an AC power cord (standard/conventional) that plugs into a standard AC power source. The AC Power Cord is interchangeable with various AC source voltages and frequencies that are within the operating range of the main power supply. A cord 103 is optionally included for connecting the hand held assembly or wand to the console; though a wireless coupling can be implemented in accordance with the level of knowledge and skill in the art.

The display screen 110 is shown as being integrated with the console 100; however, a tablet PC may be implemented which is detachable or separate from the console, or other embodiment implemented which provides a computer control for communicating signals or current to the wand for modulating or generating thermal therapy about the contact surface of the removeable applicator. A power switch 104 is optionally provided on the console, or may form part of a tablet pc.

In a general embodiment, the console is any computerized device capable of (i) receiving instructions or commands from a user via a GUI, and (ii) translating the instructions or commands into either a signal, which is communicated to the wand for producing the desired thermal output, or directly supplying current to the wand for controlling the thermal output in order to achieve the desired thermal treatment. While certain illustrative embodiments are shown and described herein, it should be appreciated that a myriad of variations may be employed by those with skill in the art to arrive at substantially the same result.

### Hand-Held Thermal Application Device; or “Wand”:

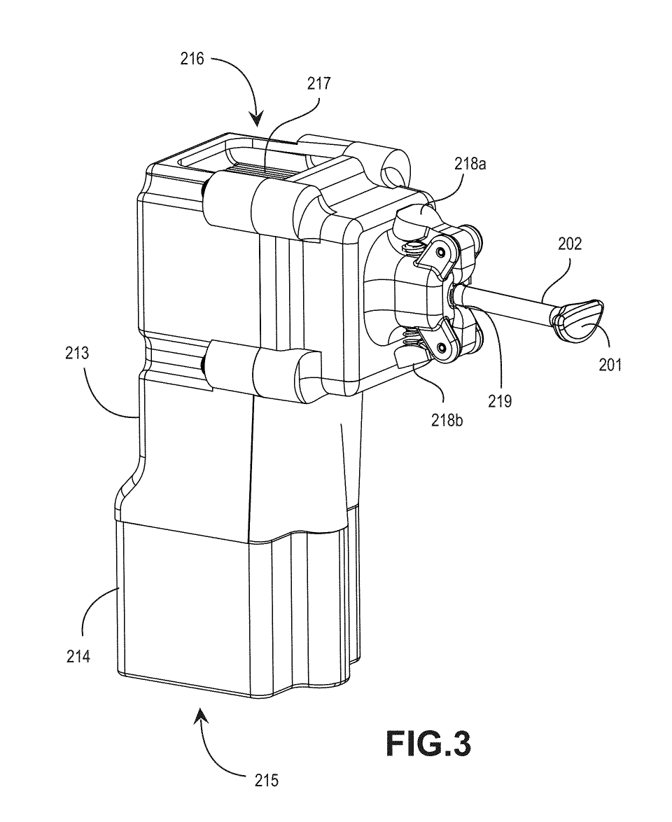

FIG. 3 shows the 3 and its elements in accordance with one embodiment. The wand comprises: a removeable thermal energy applicator 202 having a thermal contact surface 201 disposed at a distal end thereof, along with a heat exchanger shroud 213, a cooling fan housing 214, a first cooling air exit or entry location 215, a second cooling air entry or exit location 216, heat exchanger fins (also referred to as a “heat sink” 217, spring-loaded levers 218(a-b) that are configured to retain the thermal energy applicator by engaging and transmitting spring force to indents 219 in the thermal energy applicator 202. These indents 219 allow the thermal energy applicator 202 to be retained in a plurality of rotational orientations; i.e. the applicator can be rotated and seated within the interface between the applicator and an adapter-applicator interface, accordingly.

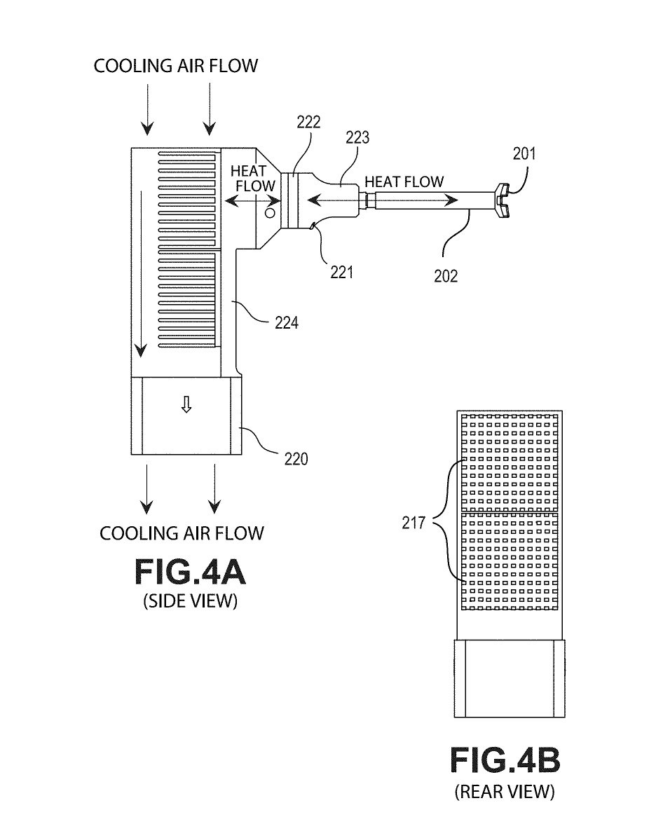

FIG. 4 shows the function of the wand 200 and key internal elements thereof. Shown again are the thermal energy applicator 202 and the heat exchanger fins 217, along with the wand cooling fan assembly 220, the temperature sensor 221, the TEC 222, the heat flow adapter 223, and the heat exchanger base 224. At the interfaces between the parts that conduct heat flow, such as between the TEC and the heat exchanger base, either thermal interface material or thermal grease is used to enhance thermal flow or conduction of thermal energy. The interface between the heat flow adapter and the thermal energy applicator may be used without thermal interface material or thermal grease to facilitate use at the expense of slightly reduced heat transfer efficiency.

In operation, electrical current delivered to the TEC in the wand causes one side of the TEC to flow heat energy outwards while simultaneously causing the other side of the TEC to absorb heat energy inwards. The direction of heat flow depends on the direction of the current flow in the TEC; i.e., the electrical polarity of the voltage applied to the TEC. Therefore, heat energy can be made to flow into the thermal energy applicator causing heating of the surface with one electrical polarity, and conversely by reversing the polarity heat energy can be made to flow into the heat exchanger thereby removing heat energy from the contact surface 201 causing cooling of the contact surface.

When the removeable thermal energy applicator 202 is being cooled, substantial heat flows into the heat exchanger and must be removed in order to keep the heat exchanger parts close to room temperature; this is necessary in order to allow the TEC to efficiently transfer heat. If the temperature of the hot side of the TEC becomes excessive, the TEC will no longer be able to transfer heat. In order to remove heat, the cooling fan assembly draws air across the heat exchanger fins, this airflow being constrained by the heat exchanger shroud.

The cooling fan speed may be actively controlled to decrease fan noise when lower levels of heat energy need to be removed. During cooling of the thermal energy applicator to the lowest temperature set point, the maximum amount of heat energy needs to be removed from the heat exchanger, so the fan can be controlled to operate at a relatively high speed. Due to the thermodynamics of the system, when the thermal energy applicator is being heated, the relative amount of heat necessary to be “added” to the heat exchanger by the airflow is minimal. Therefore, even at the highest temperature set point, the fan speed can be controlled to operate at a relatively low speed.

The temperature sensor 221 is shown located at the heat flow adapter surface, although it may be instead located at the surface of the thermal energy applicator if it is desired to control the temperature at a location farther from the TEC and closer to where the heat is conducted to and from the patient. One benefit of the latter configuration includes potentially greater temperature accuracy. However, the latter configuration presents more difficulty with temperature control due to thermal latency, and is either more complicated if the sensor is built into the thermal energy applicator given the electrical connection, or is more cumbersome for the user given that the sensor would need to be removed in order to remove the thermal energy applicator.

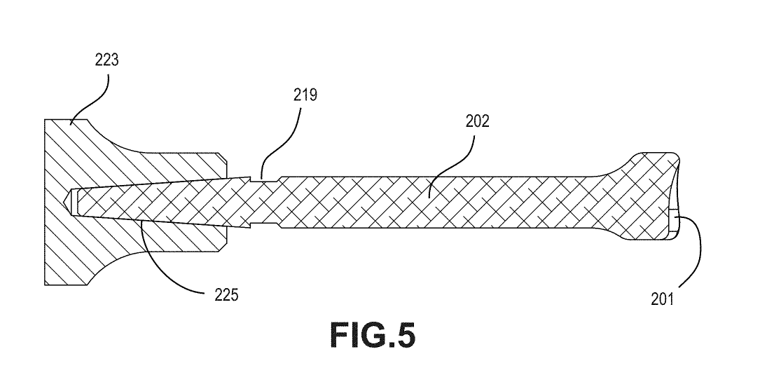

FIG. 5 shows a cross-section view of one embodiment of the heat flow adapter 223 to thermal energy applicator 202 interface (“adapter-applicator interface” or “AAI” 225). In this embodiment, the AAI interface is a tapered angle fit of small angle such that there is tight engagement of the two mating surfaces providing good surface contact for efficient heat flow. The removeable thermal energy applicator 202 is retained in its position in the heat flow adapter 223 by spring-loaded levers (See FIG. 3, 218a; 218b) pushing the surfaces together by engaging the indents 219 as described above.

In addition to the configuration shown in FIG. 5, the thermal energy applicator to heat flow adapter mating heat transfer surfaces could be a screw thread type engagement, a spring-loaded clamp type or other engagement. Also, the heat flow adapter part could be eliminated by designing the thermal energy applicator with an interface to the TEC that replicates that of the heat flow adapter part, and then by spring force or otherwise pushing the thermal energy applicator and the TEC surfaces together directly. Although the latter could offer better heat transfer efficiency, the design is difficult to implement due to the fragility and surface pressure requirements of the TEC.

Another embodiment (not shown) may include the wand with a differently shaped and sized thermal energy applicator installed. Here, the patient end of the part may be a very small, thin, flat surface with the main flat surface oriented at an angle relative to the primary axis of the part of up to ninety degrees (perpendicular). Thermal energy applicators with shape and size similar to this will be suitable for eye therapies involving thermally-augmented debridement.

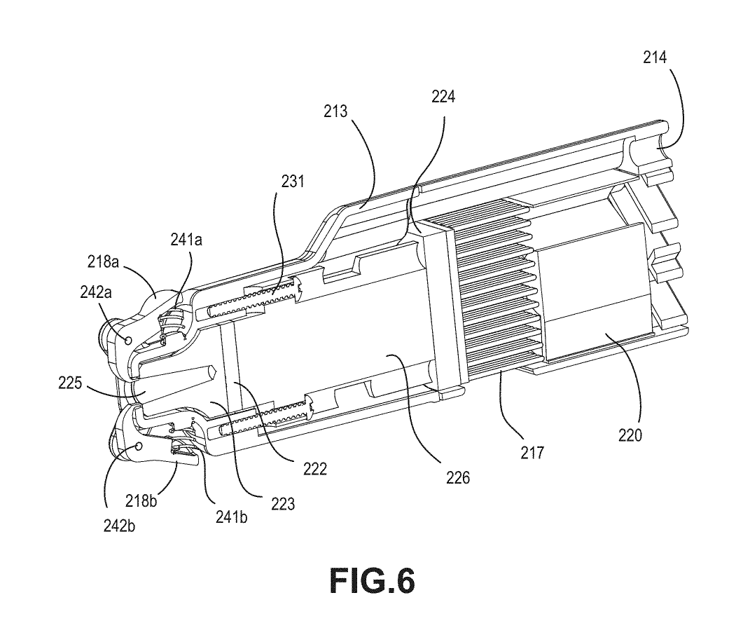

Now turning to FIG. 6, a wand is shown in accordance with another embodiment, wherein the wand is configured to produce heat or cooling of the contact surface for providing either hot or cold thermal therapy at an instant in time, or for providing modulations of both hot and cold thermal therapy according to a program administered over a period of time. The wand 200 in accordance with the embodiment illustrated in FIG. 6 comprises an assembly housing 213 housing components of the wand. The wand includes a mechanism for removeably engaging a removeable thermal applicator, the mechanism is shown including a pair of spring loaded levers, each lever 218a; 218b is attached to the housing at a pin 242a; 242b, and configured to rotate with a spring bias imparted by springs 241a; 241b. In a center of the mechanism for engaging the applicator is disposed an adapter-applicator interface 225, which is shown as a tapered cone for receiving a corresponding surface of the applicator. Screws 231 or other engagement members may be used to attach several components of the wand, especially the thermal energy components such as the heat flow adapter 223, which is coupled to a Peltier module 222, and a heat block 226 which is further coupled to the Peltier module 222 at a side opposite the heat flow adapter. The heat block is shown arranged adjacent to a heat sink 217 and fins thereof, and a fan assembly 220 is further positioned adjacent to the heat sink for cooling the fins. A fan shroud is configured to allow air to pass through the fan, cool a surface of the heat sink, and pass through an aperture adjacent the heat sink or elsewhere as may be desirable for efficient cooling.

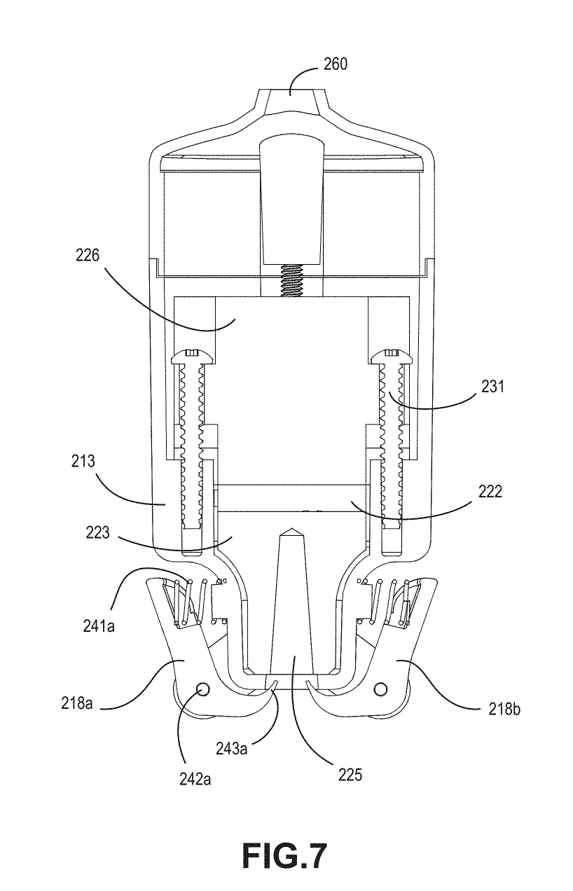

Alternatively, in accordance with another embodiment as illustrated in FIG. 7, a wand may be configured for heat only. This wand may be the only wand associated with the system, or may be provided in combination with the wand as shown in FIG. 6 wherein two wands are connected to the console, for example, one wand used for heat and another for cooling. In the embodiment of FIG. 7, the wand 200 comprises: (i) a mechanism for removeably engaging a removeable thermal applicator, the mechanism is shown including a pair of spring loaded levers, each lever 218a; 218b is attached to the housing at a pin 242a; 242b, and configured to rotate with a spring bias imparted by springs 241a; 241b, the levers further comprising teeth 243a for engaging a surface of the applicator; (ii) an assembly housing 213 for housing components of the wand; and (iii) a combination of heating components, including: a heat flow adapter having an adapter-applicator interface 225 as shown, a Peltier module 222, and a heat block 226 each arranged in series. Certain heating components can be connected using screws 231. The wand can comprise a cord opening 260 positioned at a rear side and through an axial center of the wand as shown.

In various embodiments as illustrated in FIGS. 6-7, the wand is configured to receive instructions or current from the console, and produce heat or cooling of the removeable insert engaged with the wand, such that hot, cold, or hot and cold thermal therapy may be provided to a patient.

### Removeable Thermal Therapy Applicator:

As disclosed above, one of a plurality of possible thermal energy applicators can be implemented with the wand of the system depending on the treatment requirements.

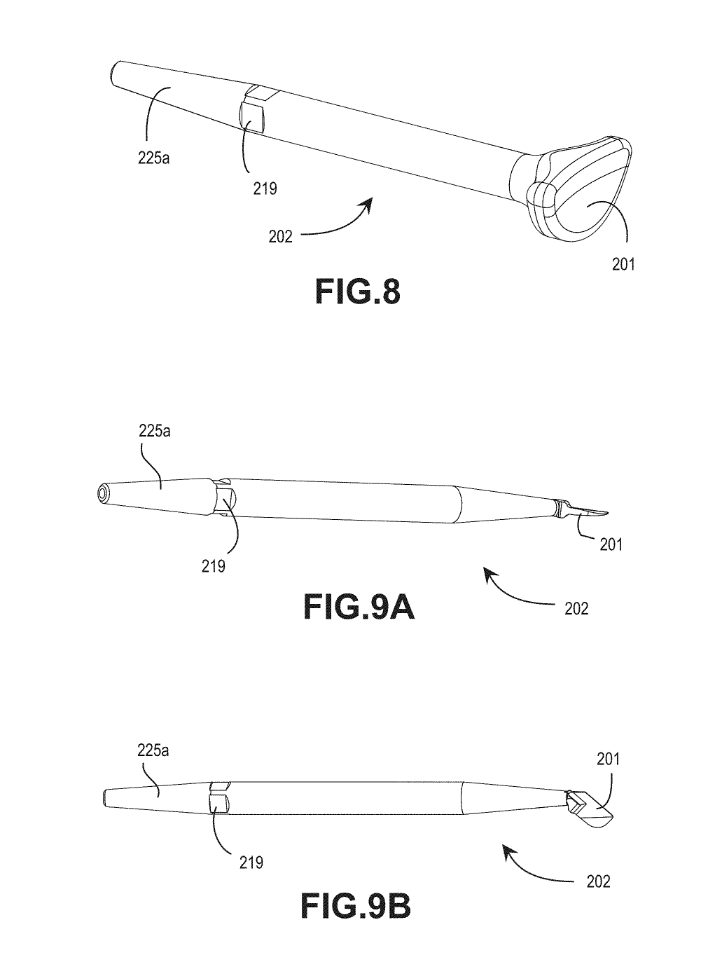

FIG. 8 shows an applicator 202 in accordance with a first embodiment, wherein the applicator comprises a tapered interface 225a configured to engage and mate with a corresponding surface of an adapter-applicator interface of a heat flow adapter, an indent 219, and a contact surface 201. Note the indent is shown as a circumferential indent; however, other indents may be similarly implemented as shown in the following illustrations (See FIGS. 9A-9B). In this particular embodiment, the contact surface 201 is adapted to contact the eyelids of a patient and apply to the eyelids a thermal therapy treatment.

FIGS. 9(A-B) show another removeable applicator 202 for use with the system, the applicator comprises a debridement tool. The debridement tool includes the same key features of most applicators for use with the system, such as a corresponding interface 225a, indent(s) 219, and a contact surface 201. Here, the contact surface resembles a knife for debridement applications. Also, here is shown multiple indents such that the tool can be rotatably arranged within the AAI of the wand.

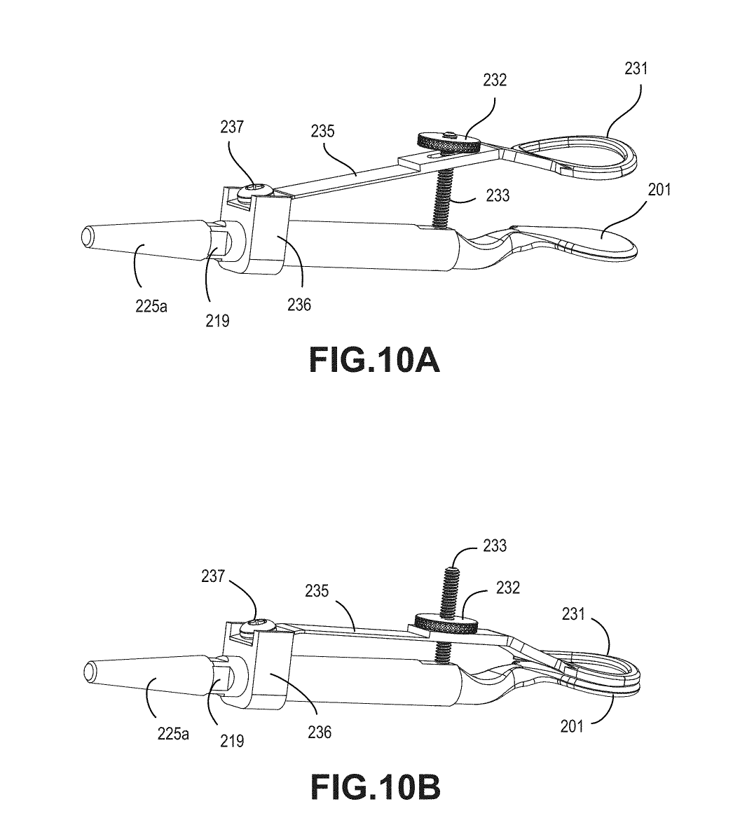

FIGS. 10(A-B) show another removeable applicator 202 for use with the system, the applicator comprises a chalazion clamp. The chalazion clamp applicator includes the same key features of most applicators for use with the system, such as a corresponding interface 225a, indent(s) 219, and a contact surface 201. However, here the chalazion applicator further comprises an annular arm 231 configured to clamp about the contact surface 201, wherein a threaded post 233 is engaged with the applicator 202 at a body thereof, for example by threading into a drilled and tapped hole of the applicator body as shown, and a nut with a textured surface for hand-tightening the nut about the threaded post. The nut is used to clamp the annular arm by varying a distance between the arm body 235 thereby changing a distance between the annular arm 231 and the contact surface 201. The arm 235 is shown engaged with a base 236 via a screw 237. The combination of components illustrated can be said to collectively form an “applicator assembly”. Using the chalazion applicator, a patient with a chalazion can be treated with cooling therapy and pressure using the annular arm and paddle forming the chalazion applicator. FIG. 10A shows the chalazion applicator in an open configuration, whereas FIG. 10B shows the chalazion in a closed configuration.

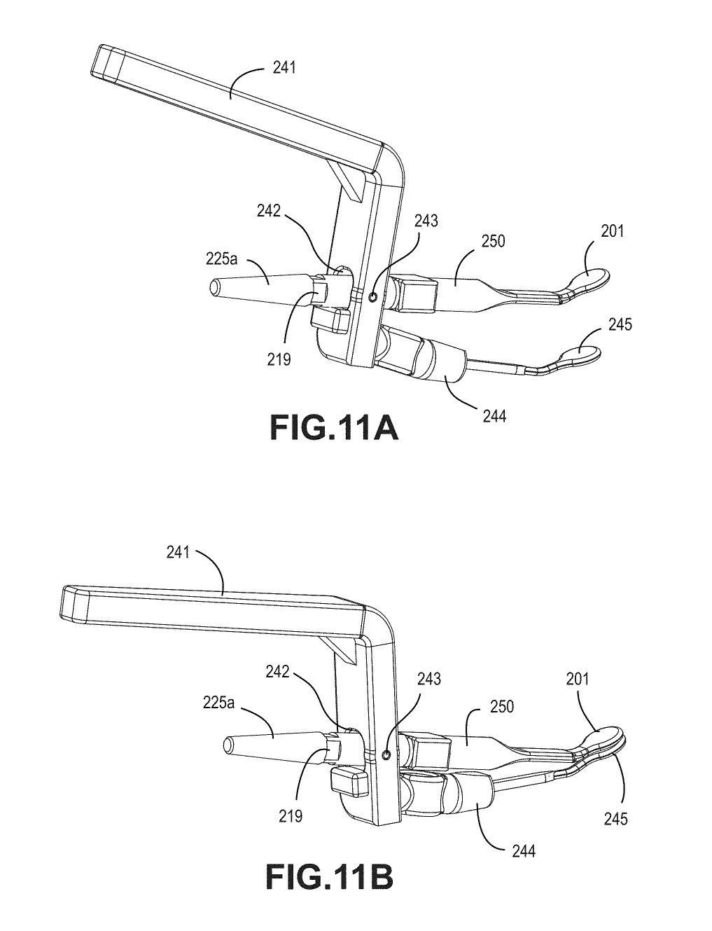

FIGS. 11(A-B) show another removeable applicator 202 for use with the system, the applicator comprises a paddle clamp. The paddle clamp applicator includes the same key features of most applicators for use with the system, such as a corresponding interface 225a, indent(s) 219, and a contact surface 201. However, here the paddle clamp applicator further comprises a first paddle 201 coupled at a distal end of a primary body 250 associated with the applicator 202, wherein the primary body 250 extends from the tapered interface 225a to the first paddle 201. A handle 241 is coupled to the primary body 250 of the applicator at a pin 243, the handle comprises an aperture 242 wherein the primary body 250 of the applicator is extended therethrough. Further coupled to the handle 241, at a side opposite a lever portion thereof, is a secondary body 244 and a second paddle 245 coupled at a distal end thereof. Here, the handle is configured to be actuated at the pin 243, such that the second paddle 245 can approach the first paddle 201 from a retracted position (FIG. 11A) to an adjacent position (FIG. 11B). While the arrangement of features are shown, it will be understood that minor variations can be similarly implemented to obtain substantially the same results.

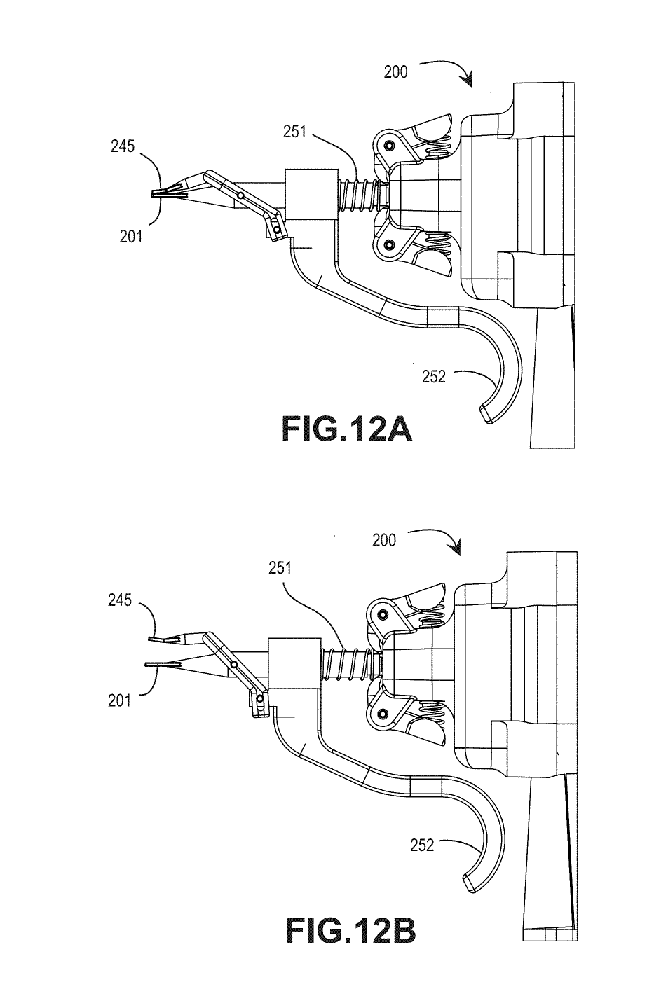

FIGS. 12(A-B) show the system with an integrated thermal energy applicator assembly, the applicator assembly comprises paddles for paddle expression, wherein the paddles are configured for actuation upon translation of a handle. Here, FIGS. 12(A-B) shows the wand 200 with a distinct version of thermal energy applicator installed, this being a manually-actuated mechanism that allows for the compression of heated/cooled paddles to aid in the forced expression of fluid from the eyelid or for similar therapies. FIG. 12A shows the mechanism in the closed position. FIG. 12B shows the mechanism in the open position. In the embodiment shown, first paddle 201 is stationary, while the second mating paddle part 245 moves either open due to spring 251 force alone acting through the trigger 252 or closed due to the user applying force to move the trigger against the spring force. Both the first and second paddles are made of thermally-conductive material. Heat transfer from the first paddle to the second paddle may be facilitated by holding the paddles together for a short period of time immediately prior to use.

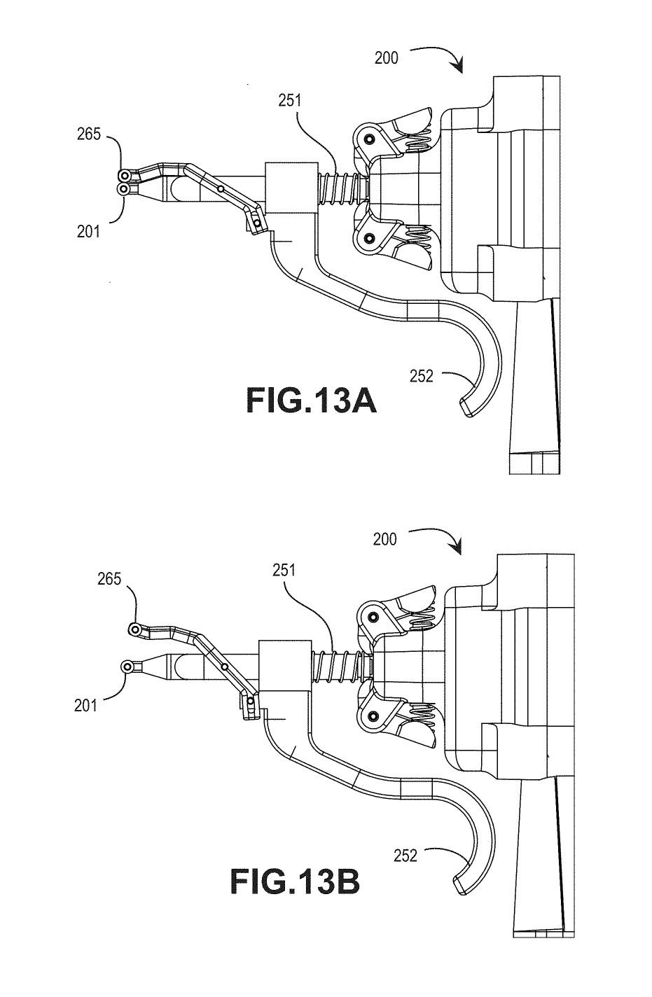

FIGS. 13(A-B) show the system with an integrated thermal energy applicator assembly, the applicator assembly comprises rollers for roller expression, wherein the rollers are configured for actuation upon translation of a handle. FIGS. 13(A-B) show the wand 200 with another version of thermal energy applicator installed, this being a manually-actuated mechanism that allows for the compression of heated/cooled rollers to aid in the forced expression of fluid from the eyelid or for similar therapies. This mechanism uses the same trigger and spring as shown in FIGS. 12(A-B). FIG. 13A shows the mechanism in the closed position. FIG. 13B shows the mechanism in the open position. In the embodiment shown, the first roller part 201 is stationary, while the second roller part 265 moves either open due to spring force alone acting through the trigger 252 or closed due to the user applying force to move the trigger against the spring force. Both first and second roller parts are made of thermally-conductive material. Heat transfer from the first roller part to the second roller part may be facilitated by holding the rollers together for a short period of time immediately prior to use. The trigger part 252 is made of a thermally non-conductive material such as plastic as heat transfer through this trigger part is undesirable.

### System Graphical User Interface (GUI):

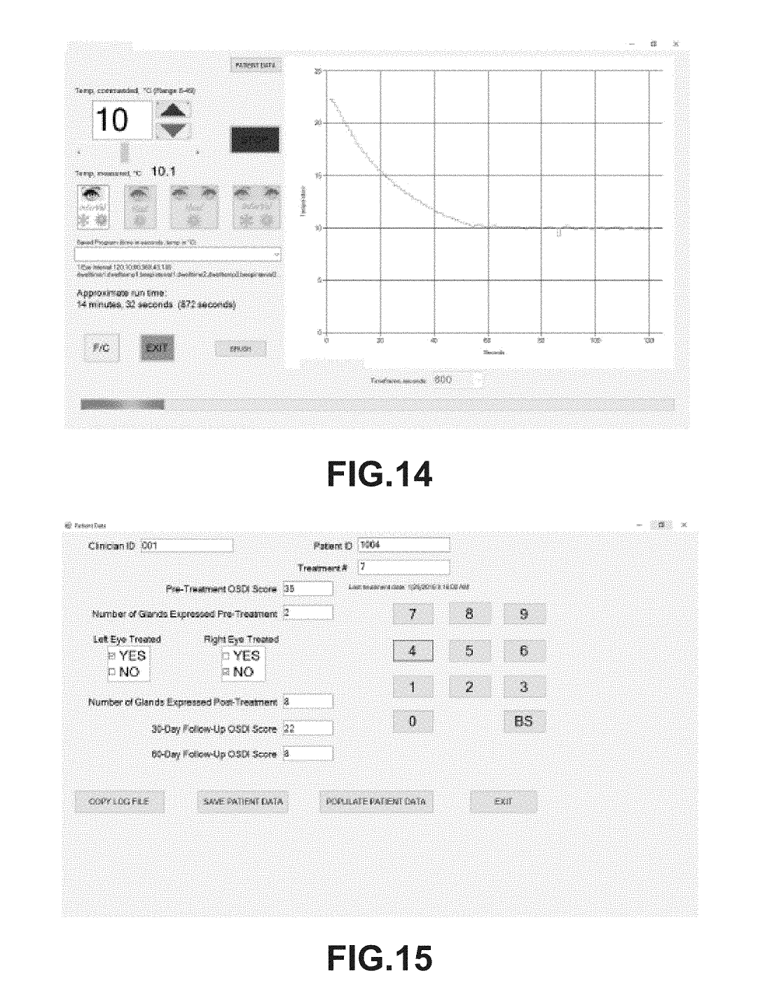

FIG. 14 shows an example of a graphical user interface (GUI) for displaying on the display screen 110 of the system (See FIG. 1), the GUI permits user-interaction for selecting and customizing a thermal therapy procedure, or for other operations inputs and interactions. Here is shown an example of the computer GUI during typical operation of the device, as during a therapy session. This screen shot example shows the display of temperature set point and measured temperature, a moving chart display of measured temperature, various time/temperature interval programs that may be selected, created or edited, and a progress bar showing an approximation of program duration remaining.

FIG. 15 shows another example of the GUI associated with the system, wherein information specific to a patient and treatment procedure are input for storage. Here, data such as clinician identifier, patient identifier, treatment iteration, and other information is entered and stored. Upon saving patient data, this data entered is configured to be stored on a database, either locally within the computer of the console, or via an internet connection, for example on the cloud. This drawing represents an example of the computer Graphical User Interface (GUI) during use of the device for patient data logging, retrieving, storing or other manipulations. This screen shot example shows identification of the clinician and patient, as well as various patient data parameters that are editable by the user. In this manner, the device may serve as a “base station” for patient information with the ability to save or retrieve information to and from a user's network, thumb drive, cloud storage space, etc.

### Interval Thermal Therapy:

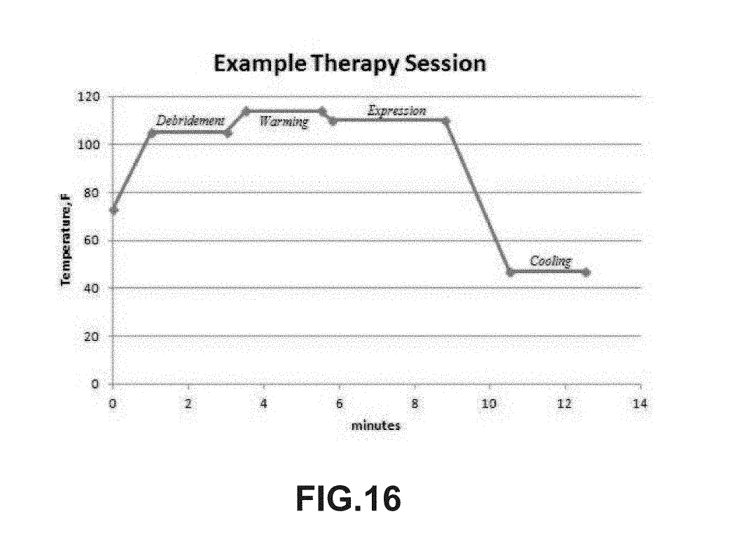

FIG. 16 shows a plot of an example therapy session which can be provided using the interval thermal therapy system. Here, the therapy session begins with initial heating from about 70° F. to about 105° F. over a duration of about one minute, followed by debridement performed at about 105° F. over about a two minute duration, thereafter followed by about three minutes of warming at about 110° F., thereafter followed by expression for a duration of about three minutes at a temperature of about 105° F., and subsequently followed by cooling the wand and applying cooling for about two final minutes at a temperature of about 50° F. While the illustrated example is suggested, it should be understood that temperatures and durations, as well as the number and order of treatments, can individually be modulated by a practitioner to arrive at a multitude of possible thermal therapy regimes.

For purposes herein, the tern “about” with respect to a temperature means plus or minus 5° F.

### System Arrangement and Assembly:

The console is constructed primarily of available off-the-shelf components: computer, power supply, analog and digital conversion board with serial port or other computer interface with or without a dedicated microprocessor, TEC controller, power entry module, power cord, wand cable, electrical connectors, inductors, capacitors, resistors, wires, fasteners, feet, etc. Custom parts include enclosure and internal bulkhead parts that are sheet metal of basic aluminum or steel alloy, machined basic aluminum alloy parts and injection molded and/or formed plastic parts.

The computer operating system is off-the shelf and the software GUI applications are custom-written for specific device uses. If used, the microprocessor firmware is also custom-written.

The wand is constructed of available off-the-shelf parts including the TEC, heat exchanger fins, cooling fan, springs (if used), wires, connectors, resistors, thermal sensor(s), thermal cutoff fuse, thermal interface materials or thermal grease, fasteners, etc. Custom parts include several injection-molded plastic parts, the internal machined or cast metal parts, and the machined or otherwise formed thermal energy applicator.

The metal parts that conduct heat may be constructed from any metal that has high thermal conductivity. These include several aluminum alloys such as alloys 6101 and 6063 which are relatively inexpensive and light and may be thinly plated with nickel to provide enhanced corrosion resistance. Silver is the best metal for thermal conductivity and may be used to obtain maximum heat transfer at the expense of higher material cost. Pure copper is an excellent material and may likewise be used at the expense of higher machining costs.

Parts may be assembled by hand and/or by automated means. Parts that are connected to each other are done so using any combination of the conventional mechanical fastening techniques (e.g., screws, pins, etc.). Printed circuit boards are constructed per typical commercial manufacturing methods. Operations such as soldering are conventionally performed using standard tools.

What is claimed is:

1. A system for providing interval thermal therapy, the system comprising:
a console coupled to a hand-held thermal application assembly;
the console including:
a computer housed in a console housing, and
a display screen;
the hand-held thermal application assembly including:
a heat flow adapter coupled to a Peltier device,
the heat flow adapter characterized by having an adapter-applicator interface, wherein at least a portion of a removeable thermal energy applicator is configured to be received and engaged with the heat flow adapter at the adapter-applicator interface; and
the removeable thermal energy applicator comprises at least one contact surface configured to communicate thermal energy between the applicator and a treatment side of a patient.

2. The system of claim 1, wherein the hand-held thermal application assembly further comprises a heat block coupled to the Peltier device at a side opposite of the heat flow adapter.

3. The system of claim 2, wherein the hand-held thermal application assembly is configured to communicate heat to the treatment site by way of the contact surface of the removeable thermal energy applicator.

4. The system of claim 2, further comprising: a heat sink coupled to the heat block, and a fan assembly configured to communicate a flow of air over the heat sink.

5. The system of claim 4, wherein the hand-held thermal application assembly is configured to communicate one of: heat and cooling to the treatment site by way of the contact surface of the removeable thermal energy applicator.

6. The system of claim 1, wherein the system further comprises a graphical user interface, wherein the graphical user interface is adapted to receive operator instructions for selecting a treatment for administration to the treatment site of the patient.

7. The system of claim 6, wherein the treatment includes at least one temperature over at least one duration.

8. The system of claim 6, wherein the treatment includes two or more temperatures, with a first temperature applied over a first duration, and a second temperature applied over a second duration, wherein each of the first and second temperatures and the first and second durations are distinct from one another.

9. The system of claim 1, wherein the removeable thermal energy applicator comprises a contact surface oriented orthogonal with respect to a body of the applicator.

10. The system of claim 1, wherein the removeable thermal energy applicator comprises a debridement tool.

11. The system of claim 1, wherein the removeable thermal energy applicator comprises thermal rollers.

12. The system of claim 1, wherein the removeable thermal energy applicator comprises a chalazion clamp.

13. The system of claim 1, wherein the removeable thermal energy applicator comprises paddles.
